(19) United States
(12) Patent Application Publication
(10) Pub. No.: US 2009/0264379 A1
(43) Pub. Date: Oct. 22, 2009

(54) Stable aqueous solution of a polyene fungicide

(75) Inventors: Ben Rudolf de Haan, Voorburg (NL); Jacobus Stark, Rotterdam (NL); Hong Sheng Tan, Bleiswijk (NL)

Correspondence Address: NIXON & VANDERHYE, PC, 901 NORTH GLEBE ROAD, 11TH FLOOR, ARLINGTON, VA, 22203, US

(73) Assignee: DSM IP ASSETS B.V., Heerlen (NL)

(21) Appl. No.: 12/453,644

(22) Filed: May 18, 2009

Related U.S. Application Data
(62) Division of application No. 10/548,272, filed on Sep. 7, 2005, now abandoned.

(30) Foreign Application Priority Data
Mar. 21, 2003 (EP) 03100727.1

(51) Int. Cl. A61K 31/7048; A01P 3/00

(52) U.S. Cl. 514/28

(57) ABSTRACT

The invention provides an aqueous composition comprising a dissolved polyene fungicide and a solubilizer, wherein at least 100 ppm of polyene fungicide is present as dissolved polyene fungicide. The aqueous compositions of the invention may further comprise a chelating and/or antioxidation agent. The compositions of the invention provide dissolved polyene fungicides in a more stable form. The present invention further provides uses of the compositions of the inventions and methods of making such compositions.

### FIELD OF THE INVENTION

The present invention relates to an aqueous solution of a polyene fungicide.

### BACKGROUND OF THE INVENTION

Polyene fungicides are used in the prevention of fungal and yeast growth in a wide variety of applications. In particular polyene fungicides, such as natamycin, are used to prevent spoilage of food products such as cheese, sausages, fruit products and beverages. Agricultural, veterinarian and pharmaceutical applications of polyene fungicides are also known.

For convenient use of preparations comprising a polyene fungicide, the activity of the polyene fungicide in the preparation should be sufficiently stable to allow handling, shipment and/or storage of the preparation. The activity of the polyene fungicide in the preparation depends on a number of different known factors, depending on the kind of polyene fungicide and the specific properties of other components in the preparation. For instance, it has been shown that exclusion of light and high concentrations in combination with moderate temperatures have a positive influence on the stability of natamycin and therefore the efficacy the polyene fungicide. The positive influence of high concentrations on the stability of the polyene fungicide is not surprising because most of the fungicide will be present in the solid state. Protection of natamycin from oxidation and ultraviolet light can be obtained by the use of chlorophyllin and other compounds (Antibiotics and Chemotherapy 9: 327-332).

The efficacy of a polyene fungicide preparation is in general determined by the concentration of the fungicide and the stability of this polyene fungicide under these conditions. Since only the dissolved fraction has antifungal activity, the fungicidal effect mostly depends on the amount of the dissolved part of the polyene fungicide, which is low in aqueous systems (most food products) and in most organic solvents.

Further, the stability is also directly linked to the solubility of the polyene fungicide. The dissolved fraction of the polyene fungicide is more susceptible to degradation by factors affecting its stability.

For many applications the low solubility of natamycin is an advantage rather than a disadvantage because most of the food spoilage fungi are susceptible to very low concentrations of natamycin. The undissolved natamycin thus forms a depot or source by compensating for the dissolved part of the fungicide which has disappeared e.g. by carrying out its activity, decomposition and/or diffusion. This is especially true when surface treatment is employed with the aim of protecting the product, such as cheese or sausages, for a longer period of time.

For many other applications a higher amount of dissolved natamycin can be advantageous. For instance when a high antifungal activity is required for a short term. This might be the case during some stages of the foodstuff production process; e.g. just before closing the packaging of the product or, in the case of the production of cheese and sausages, shortly after the production when the humidity is high and the product is more susceptible to fungal spoilage.

A high amount of dissolved natamycin is also advantageous when one has to deal with fungal species with a higher tolerance towards the fungicide. In such cases, a higher amount of the fungicide in the active (dissolved) form is required in combination with a good stability of the active form. For instance, Penicillium discolor is a species with a higher tolerance towards natamycin than the usual spoilage fungi present in cheese warehouses. High contamination levels of such a species may lead to spoilage problems because less than 40 ppm dissolved natamycin in aqueous systems might be too low to prevent the outgrowth of this mould species. The amount of dissolved polyene fungicide can be improved, for instance, by using a low or a high pH. However, the shelf life of such preparations is very limited because of the poor stability of dissolved natamycin especially at low and high pH. Therefore up to now such preparations of dissolved natamycin have to be prepared just before use. An extra disadvantage of such a practice is the need to have specialized equipment and ingredients to hand for making the preparations. A further disadvantage of a solution having a high or a low pH is that its pH is influenced by the pH of the treated subject. For example, the pH of cheese is about 5.0. This means that most of the dissolved natamycin will crystallize shortly after use on the cheese and therefore the protection against the mould will be diminished.

U.S. Pat. No. 6,369,036 describes the preparation of polyene fungicide compounds, which have an improved release of the fungicide. The enhanced activity of these preparations results from the polyene fungicide being in special crystal forms which have a high potential for dissolving. These compounds can be used in compositions to prevent spoilage of food by fungi species which have a high tolerance towards the fungicide. However these compositions still have to be prepared just before use because the compounds will be transformed to the thermodynamically most stable and thus less soluble form when suspended in an aqueous environment. Therefore, crystal forms of natamycin are known which make a fast release of dissolved natamycin possible. However, in practice, these natamycin crystal forms do not meet the requirement for stable high concentrations of dissolved natamycin especially in aqueous environment.

### SUMMARY OF THE INVENTION

The invention provides an aqueous composition which comprises a dissolved polyene fungicide and a solubilizer, wherein at least 100 ppm of polyene fungicide is present as dissolved polyene fungicide.

The invention also provides the use of an aqueous composition on or in feed, food or agricultural products.

The invention further provides a method for preserving the activity of natamycin in an aqueous solution wherein at least 100 ppm of natamycin is dissolved, the method comprising providing said solution with a solubilizer.

### DESCRIPTION OF THE INVENTION

The present invention provides a method of preparing a stable aqueous solution of polyene fungicides, which can be used directly and/or as a stock solution.

We have found that the solubility of polyene fungicides in aqueous systems can be improved by the addition of a solubilizer, preferably a surfactant. Solubilizers are compounds that can effect a solubilization of an otherwise insoluble material. In general small amounts of solubilizer will be necessary to obtain the desired effect. For example 0.05 to 8 w/w % of solubilizers will give a significant positive effect on the solubility of a polyene fungicide. Surfactants are compounds which reduce interfacial tension at the boundaries between gases, liquids and solid. However because of the expected poor stability of these solutions, combinations of polyene fungicides with solubilizers may have to be used directly or shortly after preparation.

The present invention also provides a method for preserving the activity of natamycin in an aqueous solution wherein at least 100 ppm of natamycin is dissolved, comprising providing said solution with a solubilizer. The method may further comprise providing said solution with a chelating and/or an antioxidation agent wherein said chelating agent and said antioxidation agent may be the same or a different agent.

Therefore the present invention also provides an aqueous composition comprising a dissolved polyene fungicide and a solubilizer, preferably a surfactant. At least 100 ppm, typically less than 50000 ppm or preferably from 100 to 10000 ppm of polyene fungicide is present as dissolved polyene fungicide. The polyene fungicide preferably is natamycin.

Unexpectedly, it has now been found that the solubility of polyene fungicides such as natamycin in aqueous systems can be markedly improved by means of one or more solubilizers and that such a solution is markedly more stable than would have been expected. Moreover, the stability is even better when the pH of the mixture is kept at neutral values, preferably from 5 to 9, more preferably from 6 to 8 while the solubility is maintained. According to the present invention, at least 100 ppm, preferably at least 200 ppm of polyene fungicide, preferably natamycin, is present in the aqueous solution. In general the aqueous composition of the invention comprises less than 50000 ppm, preferably less than 10000 ppm, more preferably less than 1000 ppm of polyene fungicide, which is preferably natamycin.

Surfactants may be anionic, cationic, non-ionic or amphoteric. Examples of anionic surfactants are sodium lauryl sulfate, sodium dioctyl sulfo succinate and sodium dodecyl sulfate (SDS). Examples of cationic surfactants are dodecyl ammonium chloride and hexadecyl triammonium bromide. Useful nonionic surfactants may be of the hydrophilic or of the hydrophobic type or a combination thereof. Examples of hydrophilic nonionic surfactants are polyethyleneglycol-20 sorbitan monolaurate (also known as PEG-20 sorbitan monolaurate or Tween 20), PEG-20 sorbitan monostearate (also known as Tween 60) and PEG-20 sorbitan monooleate (also known as Tween 80). Examples of hydrophobic non ionic surfactants are sorbitan monolaurate (Span 20) and sorbitan monostearate (Span60). Examples of amphoteric surfactants are alkyl betaines and alkylsulfobetaines. Preferably SDS is used as surfactant.

In general 0.1 to 5.0% w/w of surfactant is used in the composition of the invention.

Other known solubilizers are for example polyvinylpyrrolidone (PVP) or lecithin.

The stability of the solubilized natamycin can be further improved by adding a chelating agent and/or an anti-oxidation agent. A chelating agent is a compound containing two or more electron donor atoms that is capable of forming coordinate bonds to a single metal atom. Usually these compounds are used to solubilize metal atoms like calcium and other heavy metals. A well known example of a chelating agent is ethylenediaminetetraacetic acid (EDTA). Anti-oxidation agents are substances that are used to slow down the reaction of organic materials with oxygen. Examples of anti-oxidation agents are butylated hydroxyanisole, riboflavin, ascorbic acid and tocopherol.

Oxidative inactivation of polyene fungicides is promoted by several metal ions like Fe(III), Ni(II) and Cr(III). This can be prevented by chelating agents like EDTA or polyphosphates. The finding that chelating agents can preserve the stability of soluble natamycin without the presence of any of these harmful heavy metal in the solution is therefore very surprising.

The chelating agent preferably comprises an aminocarboxylate, for instance ethylenediaminetetraacetic acid and N-dihydroxyethylglycine, a hydroxycarboxylate like citric acid and tartaric acid or a polyphosphate like tripolyphosphoric acid and hexametaphosphoric acid. Preferably a non-acidic chelating agent will be used.

More preferably said chelating agent comprises ethylenediamine-N,N,N′,N′-tetraacetic acid (EDTA) or a functional equivalent thereof. A functional equivalent of EDTA is a functional part, derivative and/or analogue of EDTA comprising the same fungicide preserving activity in kind not necessarily in amount. The most common functional equivalents of EDTA are the various salts of EDTA such as sodium, potassium, lithium, ammonium, calcium and/or copper salts of EDTA. However, substitution of one or more groups of the molecule with other equivalent groups are also preferred equivalents of EDTA. Non-limiting examples of such equivalents are 1,3-diamino-2-hydroxypropane-N,N,N′,N′-tetraacetic acid and 1,3-diaminopropane-N,N,N′,N′-tetraacetic acid.

Preferably the amount of chelating agent in said aqueous solution is between 10-10000 ppm. More preferably, between 20-1000 ppm, most preferably between 30-300 ppm.

For the present invention an anti-oxidation agent may also be added. The anti-oxidation agent is preferably a non-acidic anti-oxidation agent. Preferably, said anti-oxidation agent comprises ascorbic acid, citric acid, Butyl-hydroxy-anisole (BHA), Butyl-hydroxy-toluene (BHT), a gallate, a tocoferol, ascorbyl palmitate and/or calcium ascorbate. More preferably, said anti-oxidation agent comprises BHA, BHT, a tocoferol and/or a gallate.

Preferably, said anti-oxidation agent is present in said aqueous solution in an amount of 10-10000 ppm.

In another aspect, the invention provides an aqueous composition comprising a dissolved polyene fungicide, a chelating agent and/or an anti-oxidation agent, wherein said chelating agent and said anti-oxidation agent may be the same agent or a different agent. Preferably between 100 and 10000 ppm polyene fungicide is dissolved in this solution. Preferably no polymers, for example polymer beads, are present in this composition.

Advantageously the composition of the invention can be stored for at least one week without a loss of more than 10% of the polyene antifungal compound activity. More preferably, the composition may be stored for at least two weeks, more preferably at least one month and most preferably at least 3 months without a loss of more than 10% of the polyene antifungal compound.

In general the storage temperature will be between 4 and 30° C., preferably between 15 and 25° C.

In another embodiment the solution of the invention is packed in a container suitable for storage and/or shipment of said solution. Preferably, said solution can be stored for at least one week, preferably at least two weeks, more preferably at least one month and most preferably at least 3 months.

In one embodiment an aqueous solution according to the invention is obtained by a method according to the invention.

### EXAMPLES

### Material and Methods

Materials
Di-sodium-EDTA, purchased from Chemolanda by, 2596 BP Den Haag, The Netherlands.
Ammonia, purchased from Gaches Chimie France, 31750 Escalquens, France.
SDS Solution 20% (w/w), purchased by Bio-Rad Laboratories, Inc 2000 Alfred Nobel Drive Hercules, Calif. 94547 USA.
Delvocid®, containing 50% (w/w) active natamycin and 50% lactose, DSM Food Specialties, P.O. Box 1, 2600 MA, Delft, The Netherlands.
Amphotericin B code A-4888, lot 122K4013, purchased from Sigma Aldrich Chemie, GmbH, PO Box 1120 Steinheim Germany.
Nystatin code N-6261, lot 120K11351, purchased from Sigma Aldrich Chemie, GmbH, PO Box 1120 Steinheim Germany.

Analytical Method for the Determination of Amount of Natamycin

This method was used to analyze the amount of

active natamycin in a water-based mixture of several components.
The method was HPLC based using the International Dairy Federation (Provisional ADF Standard 140, 1987) with a Lichrosorb RP 8 column.
Detection was by UV at 303 nm with a range of 0.1-4 mg/L with an injection volume of 20 μl.
Sample preparation was carried out by weighing 2 gram prepared formulation with an accuracy of 1 mgram in a measuring flask.
4 ml demineralized water (demiwater) was added and the mixture was stirred for 15 minutes to get a homogeneous suspension.
Subsequently 80 ml ethanol was added and the mixture was stirred for 10 minutes.
After ultrasonic treatment the solution was filled up to 100 ml with de-minaralized water and then diluted and/or filtered (0.2 μm) before injecting.
The amount of active natamycin was calculated as ppm against a series of standards of known natamycin concentrations.

### Example 1

This example describes a method to prepare a formulation suitable for use a high soluble natamycin formulation and which was developed to test the stability in time in relation to the added ingredients and physical parameters.

The mixtures were made with an electric top stirrer, type RW 20 DZM, from Janke & Kunkel equipped with a Ruston-type stirrer.

The mixtures were made by adding 2 gram Delvocid (Natamycin) together with 50 gram 20% SDS solution and eventually additives to a final weight of 1000 gram with demineralised (=demi) water.

This crude mixture was mixed for 5 minutes to obtain a homogeneous mixture and accordingly adjusted to pH 4-6 with ammonia.

The obtained formulations with an added amount of active natamycin of 1000 ppm were stored at 18° C. in a closed pot in the dark.

The prepared mixtures were measured over time for the amount of active natamycin using the analytical method as described hereinabove.

### Example 2

The mixture as described in Example 1 was adjusted to pH=4.0 or pH=6.0 and the natamycin activity was measured over time.

The results are shown in Table 1.

TABLE 1
Rest activity of completely dissolved natamycin over time (in ppm)

| Time | pH = 4.0 | pH = 6.0 |
|---|---|---|
| 1 day | 672 | 988 |
| 3 weeks | <0.1 | 444 |
| 9 weeks | <0.1 | 57 |

### Example 3

The mixture as described in Example 1 is combined with 1000 ppm di-sodium-EDTA, adjusted to pH 6.0 and the natamycin activity was measured over time.

The results are set out in Table 2.

TABLE 2
Rest activity of completely dissolved natamycin over time (in ppm)

| Time | pH = 6.0 | pH = 6.0 + Na₂EDTA |
|---|---|---|
| 1 day | 988 | 1000 |
| 3 weeks | 444 | 805 |
| 9 weeks | 57 | 560 |

### Example 4

Mixtures are made with several polyene fungicides in several concentrations The used polyene fungicides are nystatin, amphotericin and natamycin. The solubility rate is measured visually of the polyene fungicides in demineralized water as such or according to example 1 in combination with SDS to a final SDS concentration of 1% (w/w). It is soluble if a mixture is clear after one minute stirring at room temperature.

The results are set out in Table 3

TABLE 3
Solubility of several polyene fungicides in water and water + SDS.

| | Used amount | | Solublized |
|---|---|---|---|
| | of polyene | | after 1 minute |
| Polyene | fungicide (ppm) | | stirring at |
| fungicide | in final mixture | Solvent | room temperature |
| Natamycin | 250 | Demi- Water | No |
| Natamycin | 250 | 1% (w/w) SDS | Yes |
| Natamycin | 500 | 1% (w/w) SDS | Yes |
| Natamycin | 1000 | 1% (w/w) SDS | Yes |
| Nystatin | 250 | Demi - Water | No |
| Nystatin | 250 | 1% (w/w) SDS | Yes |
| Nystatin | 500 | 1% (w/w) SDS | Almost |
| Nystatin | 1000 | 1% (w/w) SDS | No |
| Amphotericin B | 250 | Demi - Water | No |
| Amphotericin B | 250 | 1% (w/w) SDS | Yes |
| Amphotericin B | 500 | 1% (w/w) SDS | Yes |
| Amphotericin B | 1000 | 1% (w/w) SDS | Yes |

1-13. (canceled)

14. A method to enhance solubility of natamycin in an aqueous solution wherein at least 100 ppm of natamycin is dissolved, the method comprising dissolving natamycin in the aqueous solution in the presence of a surfactant.

15. (canceled)

16. A method according to claim 14, further comprising adding a chelating and/or an antioxidation agent to said solution wherein said chelating and said antioxidation agent may be the same or a different agent.

17. A method according to claim 14, wherein 200 to 50000 ppm of natamycin is dissolved.

18. A method to enhance solubility of a polyene fungicide to at least 100 ppm dissolved polyene fungicide when preparing a aqueous composition comprising the polyene fungicide, the method comprising dissolving the polyene fungicide in aqueous composition in the presence of a surfactant.

19. A method according to claim 18, wherein the amount of surfactant is 0.1 to 5% (w/w).

20. A method according to claim 18, wherein the aqueous composition comprises from 100 to 50,000 ppm dissolved polyene fungicide.

21. A method according to claim 18, wherein the aqueous composition has a pH of from 5 to 9.

22. A method according to claim 18, wherein the polyene fungicide is natamycin.

23. A method according to claim 18, wherein the aqueous composition further comprising a chelating agent and/or an anti-oxidation agent.

24. A method according to claim 23, wherein the chelating and/or anti-oxidation agent may be the same or may be a different agent.

25. A method according to claim 23, wherein the chelating agent, the anti-oxidation agent or both are non-acidic.

26. A method according to claim 23, wherein the chelating agent is EDTA or a salt thereof.

27. A method according to claim 23, wherein the amount of the chelating or the anti-oxidation agent is 10 to 10000 ppm.
